(19) United States
(12) Patent Application Publication
(10) Pub. No.: US 2020/0043053 A1
(43) Pub. Date: Feb. 6, 2020

(54) DIGITAL SIGNAGE SYSTEM WITH HAIR/SKIN CARE DIAGNOSIS FUNCTION AND METHOD FOR IMPLEMENTING THE SAME

(71) Applicants: CORUS INC., Suwon-si (KR); CORUS INC., Hoffman Estates, IL (US)

(72) Inventors: Ghaeun JEON, Hoffman Estates, IL (US); Eric JEON, Hoffman Estates, IL (US)

(21) Appl. No.: 16/051,868

(22) Filed: Aug. 1, 2018

(51) Int. Cl. G06Q 30/02; G06T 7/00; G16H 30/40; A61B 5/00; G02B 21/36

(52) U.S. Cl. CPC G06Q 30/0269; G06T 7/0012; G16H 30/40; A61B 5/0077; A61B 5/441; A61B 5/448; A61B 2576/00; G02B 21/368; G06T 2207/10016; G06T 2207/10056; G06T 2207/30088; A61B 2503/12; G02B 21/367

(57) ABSTRACT

A digital signage system with hair/skin care diagnosis function is provided that includes a microscope unit configured to scan hair/skin of a user and acquires hair/skin image and video data, a process unit coupled to the microscope unit, and configured to process the hair/skin image and video data, and a display unit coupled to the process unit, and configured to display a plurality of windows for displaying advertisements or the hair/skin image and video data and diagnosis results. When the microscope unit operates and acquires the hair/skin image and video data, the process unit switches a mode of operation of the digital signage system from a normal mode to a diagnosis mode, in which not only advertisements but also hair/skin care information related to a specific symptom of a user are displayed.

### BACKGROUND OF THE INVENTION

### Field of the Invention

The present invention relates to a digital signage system with hair/skin care diagnosis function and a method for implementing the same, and more particularly, to a digital signage system with hair/skin care diagnosis function, in which not only hair/skin care diagnosis service but also hair/skin care information related to a specific symptom of a user are provided to the user, and a method for implementing the digital signage system with hair/skin care diagnosis function.

### Description of the Related Art

With the development of the multimedia technology, digital signage has been widely used in public spaces, transportation systems, museums, stadiums, retail stores, hotels, and restaurants etc., to provide public and product information, wayfinding, exhibitions, marketing and advertising.

Some hair/skin care shops also have started to use digital signage to advertise their service and related hair/skin care products they sell in the shops. However, the advertisement contents the digital signage system displays have not been linked to a specific symptom of customers. That is, the advertisement contents are displayed on the digital signage system sequentially or randomly without linking to the specific symptom of customers, thus decreasing the effectiveness of the advertisement.

Meanwhile, hair/skin care diagnosis devices with microscope have been separately used from digital signage, for the purpose of analyzing various elements of the hair/skin, including texture, blemishes, pores, brightness, hair loss, hair weakness, hair skin trouble and coloring, and providing the diagnosis results and the progress of the care to the customers.

In this regard, there may thus be a need for a digital signage with hair/skin care diagnosis function that provides symptom-linked advertisements to a customer with specific symptom, in which not only hair/skin care diagnosis service but also hair/skin care information related to the specific symptom are provided to the customer.

### SUMMARY OF THE INVENTION

An aspect of the present invention is to solve the above-described problems and meet the need.

Another aspect of the present invention is to provide a digital signage system with hair/skin care diagnosis function, in which hair/skin care information and hair/skin care diagnosis service are provided to a user, and a method for implementing the digital signage system with hair/skin care diagnosis function.

Another aspect of the present invention is to provide a digital signage with hair/skin care diagnosis function that provides symptom-linked advertisements to a customer with a specific symptom, in which not only hair/skin care diagnosis service but also hair/skin care information related to the specific symptom are provided to the customer, thereby improving the effectiveness of the advertisement.

Another aspect of the present invention is to provide a digital signage system with hair/skin care diagnosis function capable of switching a mode of operation from a normal mode to a diagnosis mode when the digital signage system acquires the hair/skin image and video data for diagnosis, wherein in the normal mode, the digital signage system with hair/skin care diagnosis function displays advertisements only, but in the diagnosis mode, the digital signage system with hair/skin care diagnosis function displays not only the advertisements but also the hair/skin image and video data for diagnosis and the diagnosis results.

According to one or more embodiments, there is provided a digital signage system with hair/skin care diagnosis function including a microscope unit configured to scan hair/skin of a user and acquire hair/skin image and video data, a process unit coupled to the microscope unit, and configured to process the hair/skin image and video data, and a display unit coupled to the process unit, and configured to display a plurality of windows for displaying advertisements or the hair/skin image and video data and the diagnosis results. The microscope unit controls magnification and resolution of the hair/skin image and video data, and transmits the hair/skin image and video data to the process unit, and the process unit monitors a connection state of the microscope unit, receives the hair/skin image and video data from the microscope unit, stores the hair/skin image and video data, and switches a mode of operation of the digital signage system with hair/skin care diagnosis function from a normal mode to a diagnosis mode when the microscope unit operates and acquires the hair/skin image and video data. In the normal mode, the process unit transmits advertisement contents to the display unit so that the plurality of windows display advertisements, and in the diagnosis mode, the process unit transmits both advertisement contents and the hair/skin image and video data to the display unit so that one of the plurality of windows displays the hair/skin image and video data and the diagnosis results, and another window of the plurality of windows displays advertisements.

According to one or more embodiments, there is provided a method for implementing the digital signage system with hair/skin care diagnosis function, the digital signage system with hair/skin care diagnosis function including a microscope unit configured to scan hair/skin of a user and acquires hair/skin image and video data, a process unit coupled to the microscope unit, and configured to process the hair/skin image and video data, and a display unit coupled to the process unit, and configured to display a plurality of windows for displaying advertisements or the hair/skin image and video data and the diagnosis results, the method for implementing the digital signage system with hair/skin care diagnosis function including: a first operation in which the microscope unit controls magnification and resolution of the hair/skin image and video data, and transmits the hair/skin image and video data to the process unit; and a second operation in which the process unit monitors a connection state of the microscope unit, receives the hair/skin image and video data from the microscope unit, stores the hair/skin image and video data, and switches a mode of operation of the digital signage system with hair/skin care diagnosis function from a normal mode to a diagnosis mode when the microscope unit operates and acquires the hair/skin image and video data, wherein in the normal mode, the process unit transmits advertisement contents to the display unit so that the plurality of windows display advertisements, and in the diagnosis mode, the process unit transmits both advertisement contents and the hair/skin image and video data to the display unit so that one of the plurality of windows displays the hair/skin image and video data and the diagnosis results, and another window of the plurality of windows displays advertisements.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic diagram of a digital signage system with hair/skin care diagnosis function according to an embodiment of the present invention;

FIG. 2 is a schematic diagram of a process unit of the digital signage system with hair/skin care diagnosis function according to an embodiment of the present invention;

FIG. 3A is a schematic diagram illustrating a display unit of the digital signage system with hair/skin care diagnosis function and its mode switching according to an embodiment of the present invention;

FIG. 3B is a diagram illustrating a mode switching based on the state of microscope and image/video acquisition in the digital signage system with hair/skin care diagnosis function according to an embodiment of the present invention;

FIG. 4 is a schematic diagram illustrating the generation of the dynamic self-learning reference data-set and the integration with the initial factory implemented reference data-set to form the integrated reference data-set in the digital signage system with hair/skin care diagnosis function according to an embodiment of the present invention; and

FIG. 5 is a schematic diagram illustrating a scenario of operation of the digital signage system with hair/skin care diagnosis function according to an embodiment of the present invention.

### DETAILED DESCRIPTION OF THE INVENTION

In the following detailed description of the present invention, references are made to the accompanying drawings that show, by way of illustration, specific embodiments in which the present invention may be implemented. These embodiments are described in sufficient detail to enable those skilled in the art to implement the present invention. It should be understood that various embodiments of the present invention, although different, are not necessarily mutually exclusive. For example, specific features, structures, and characteristics described herein, in connection with one embodiment, may be implemented within other embodiments without departing from the spirit and scope of the present invention. In addition, it should be understood that the location or arrangement of individual elements within each disclosed embodiment may be modified without departing from the spirit and scope of the present invention. The following detailed description is, therefore, not to be taken in a limiting sense, and the scope of the present invention is defined only by the appended claims, appropriately interpreted, along with the full range equivalent to what the claims claim.

Hereinafter, embodiments of the present invention will be described with reference to the accompanying drawings, so that those skilled in the art can easily carry out the present invention.

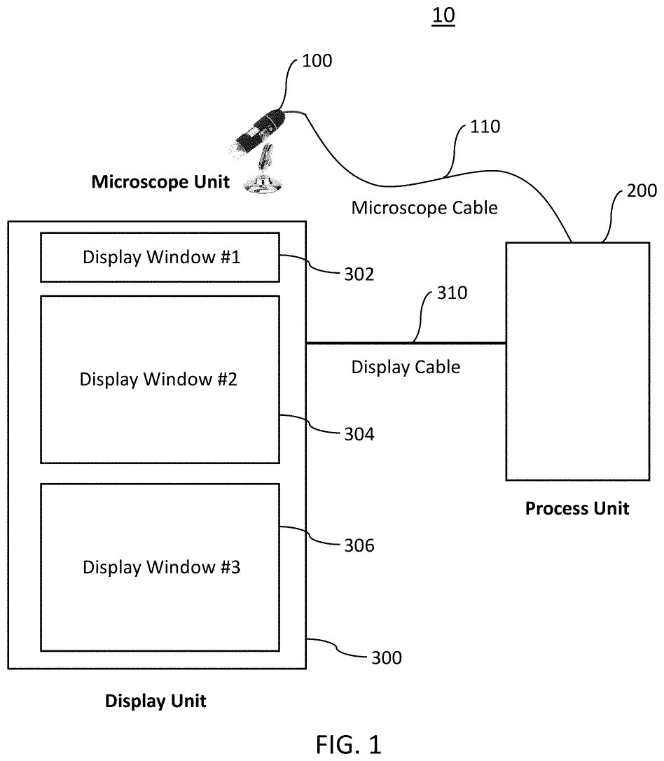

FIG. 1 is a schematic diagram of a digital signage system with hair/skin care diagnosis function 10 according to an embodiment of the present invention. Referring to FIG. 1, the digital signage system with hair/skin care diagnosis function 10 may include a microscope unit 100 configured to scan hair/skin of a user and acquires hair/skin image and video data, and a process unit 200 coupled to the microscope unit 100, and configured to process the hair/skin image and video data, and a display unit 300 coupled to the process unit 200, and configured to display a plurality of windows for displaying advertisements or the hair/skin image and video data and diagnosis results.

The microscope unit 100 may be connected to the process unit 200 with a microscope cable 110. The microscope unit 100 scans hair/skin of a user, acquires the hair/skin image and video data, controls magnification and resolution of the hair/skin image and video data, and transmits the hair/skin image and video data to the process unit 200.

The process unit 200 monitors a connection state of the microscope unit 100, receives the hair/skin image and video data from the microscope unit 100, stores the hair/skin image and video data, and switches a mode of operation of the digital signage system with hair/skin care diagnosis function 10 from a normal mode to a diagnosis mode when the microscope unit 100 operates or acquires the hair/skin image and video data. In the normal mode, the process unit 200 transmits advertisement contents to the display unit 300 so that the plurality of windows display advertisements, and in the diagnosis mode, the process unit 200 transmits both advertisement contents and the hair/skin image and video data to the display unit 300 so that one of the plurality of windows displays the hair/skin image and video data and the diagnosis data, and another window of the plurality of windows displays advertisements.

The display unit 300 may include, for example, a display window #1 302, a display window #2 304, and display window #3 306. The display unit 300 may be connected to the process unit 200 with a display cable 310.

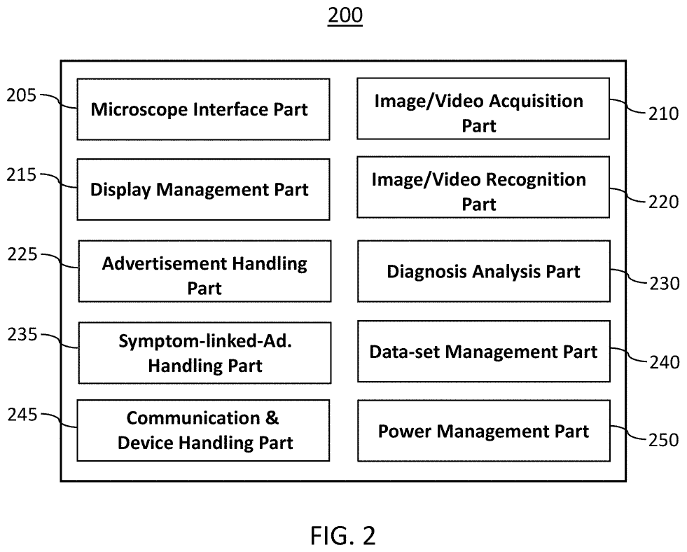

FIG. 2 is a schematic diagram of a process unit 200 of the digital signage system with hair/skin care diagnosis function 10 according to an embodiment of the present invention. Referring to FIG. 2, the process unit 200 may include a microscope interface part 205, an image/video acquisition part 210, a display management part 215, an image/video recognition part 220, an advertisement handling part 225, a diagnosis analysis part 230, a symptom-linked advertisement handling part 235, a data-set management part 240, a communication and device handling part 245, and a power management part 250.

The microscope interface part 205 manages the connection to the microscope unit 100 and transmits the image and video data received from the microscope unit 100 to the image/video acquisition part 210. In addition, in order to automatically switch the mode of operation of the digital signage system from the normal mode to the diagnosis mode when the microscope unit 100 operates, the microscope interface part 205 monitors the on/off state of the microscope unit 100 and transmits the state to the display management part 215.

The image/video acquisition part 210 stores the image and video data for a customer's hair/skin care diagnosis in a file format into a memory allotted for the customer and transmits to the image/video recognition part 220 after converting the image and video data into a file format effective for image/video recognition. Also, in order to automatically switch the mode of operation of the digital signage system from the normal mode to the diagnosis mode, the image/video acquisition part 210 monitors whether image and video data is valid or idle and transmits the valid/idle status to the display management part 215.

The display management part 215 switches from the normal mode to the diagnosis mode and vice versa, according to on/off status of the microscope unit 100 or valid/idle status of image/video data inputted from the microscope unit 100. The display management part 215 also manages and operates the plurality of windows of the display unit 300 for each mode.

The image/video recognition part 220 analyzes the status of image and video data received from the image/video acquisition part 210, recognizes symptoms on the hair and skin of the customer, quantifies the recognition results, and stores the quantification results in a symptom category information table which includes symptoms categorized according to various elements of the hair/skin, such as texture, blemishes, pores, brightness, hair loss, hair weakness, hair skin trouble and coloring. Detailed information on the process and the results of the image/video recognition is transmitted to the diagnosis analysis part 230 and summarized information is transmitted to the symptom-linked advertisement handling part 235.

The advertisement handling part 225 manages and operates general advertisement contents which are performed in the normal mode.

The diagnosis analysis part 230 stores the detailed information on the process and the results of the image/video recognition received from the image/video recognition part 220 in a database managing the history of hair and skin diagnosis of a customer and analyzes the status of the diagnosis and degree of progress in hair/skin care based on the accumulated data. In addition, the diagnosis analysis part 230 processes the analyzed information into a useful format to the customer and provides the processed information.

The symptom-linked advertisement handling part 235 manages and operates advertisement contents which are performed in the diagnosis mode. The symptom-linked advertisement handling part 235 displays products or service related to the symptom of the customer in an advertising window according to the summarized information of image/video recognition received from the image/video recognition part 220 while the customer is taking a hair/skin care diagnosis.

The data-set management part 240 stores and manages, for the image/video recognition, a reference data-set consisting of a static reference image/video data-set and a dynamic reference image/video data-set. The static reference image/video data-set refers to an initial and factory implemented data-set and is provided from the manufacturer when the digital signage system with hair/skin care diagnosis function is released from the factory. The dynamic reference image/video data-set refers to a reference data-set obtained by processing the hair/skin image and video data collected from service locations where the digital signage system with hair/skin care diagnosis function 10 is operated. The data-set management part 240 improves the quality, i.e., the degree of recognition of image/video data by integrating the initial reference data-set and the dynamic reference data-set generated during the service.

The communication and device handling part 245 receives advertisement contents or provides diagnosis results to the customer through communication with a client node or an external server. A mobile app or computer equipment may be used as an external device.

The power management part 250 manages the power that is supplied to the display unit 300 and the microscope unit 100, according to the operation of the digital signage system with hair/skin care diagnosis function 10.

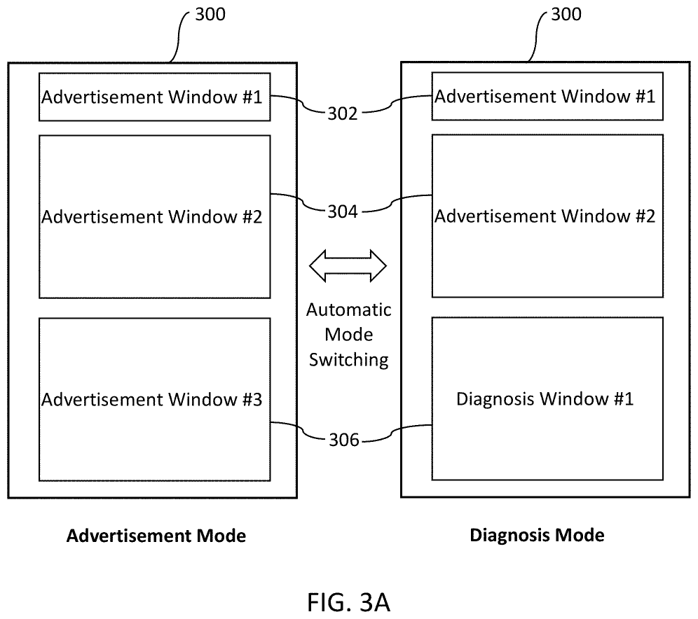

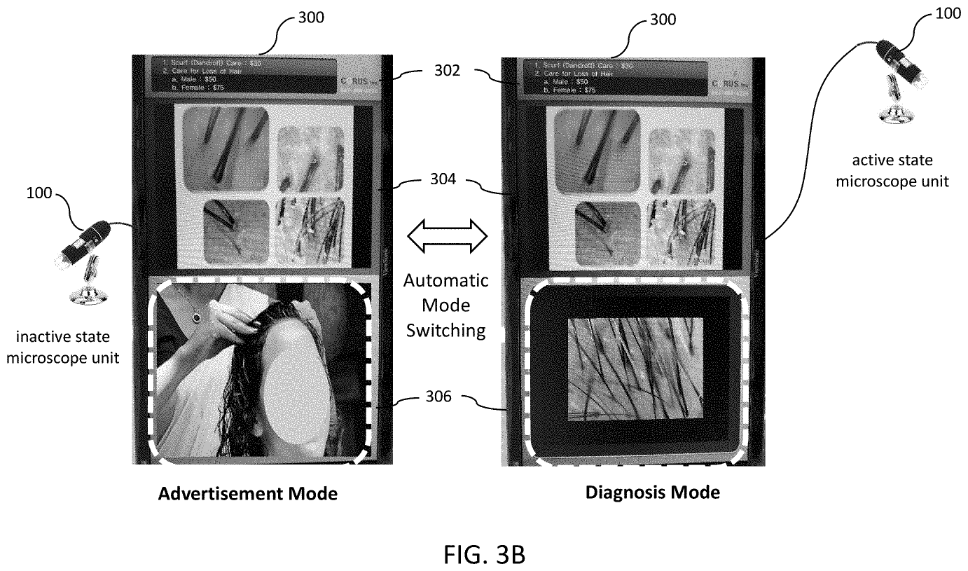

FIG. 3A is a schematic diagram illustrating a display unit 300 of the digital signage system with hair/skin care diagnosis function 10 and its mode switching according to an embodiment of the present invention.

Referring to FIG. 3A, a mode of operation of the display unit 300 is changed from the normal mode to the diagnosis mode when the microscope unit 100 operates or acquires the hair/skin image and video data, wherein in the normal mode, the display unit 300 displays advertisements only, but in the diagnosis mode, one window 306 of the plurality of windows of the display unit 300 displays the hair/skin image and video data for diagnosis and the diagnosis results and two other windows 302, 304 of the plurality of windows display advertisements.

FIG. 3B is a diagram illustrating a mode switching based on the state of microscope and image/video acquisition in the digital signage system with hair/skin care diagnosis function 10 according to an embodiment of the present invention.

Referring to FIG. 3B, while the customer is taking hair/skin care diagnosis service, the display unit 300 displays products or service related to the symptom of the customer in an advertising window 304 according to the summarized information of image/video recognition received from the image/video recognition part 220. That is, the digital signage system with hair/skin care diagnosis function 10 according to an embodiment of the present invention provides symptom-linked advertisements to a customer with a specific symptom, in which not only hair/skin care diagnosis service but also hair/skin care information related to the specific symptom are provided to the customer, thereby improving the effectiveness of the advertisement.

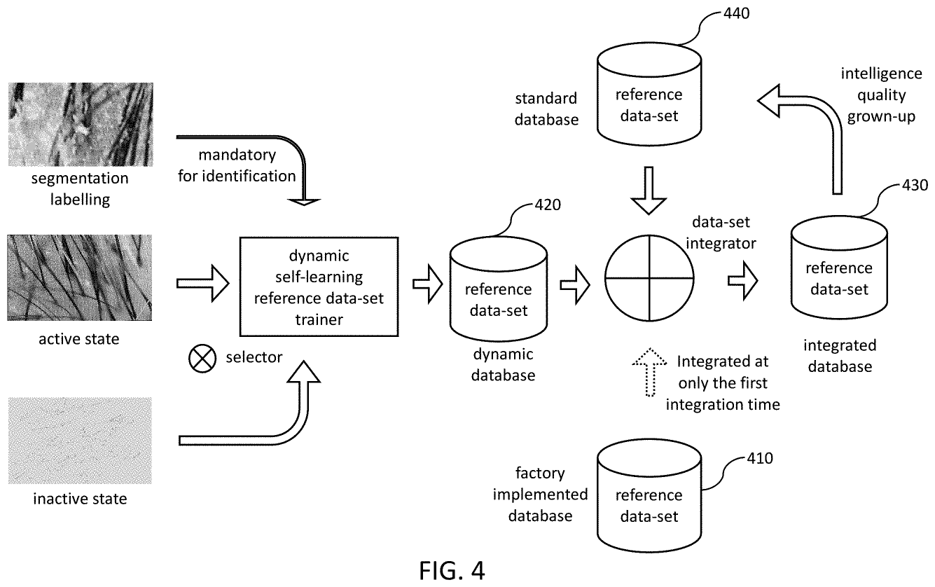

FIG. 4 is a schematic diagram illustrating the generation of the dynamic self-learning reference data-set and the integration with the initial factory implemented reference data-set to form the integrated reference data-set in the digital signage system with hair/skin care diagnosis function according to an embodiment of the present invention.

Referring FIG. 4, the image/video recognition reference data-set are collected according to the identification which categorizes the reference data-set into the initial reference data-set 410, the dynamic self-learning reference data-set 420, and the integrated reference data-set 430 generated by integrating the initial reference data-set 410 and the dynamic self-learning reference data-set 420. A standard database is built by integrating all the reference data-sets and the reference data-set 440 of the standard database is used in the process of the image/video recognition. The reference data-set 440 of the standard database is grown up through the regeneration of the dynamic reference data-set 420 and feedback process.

The initial reference data-set 410 and the dynamic self-learning reference data-set 420 may be identified by segmentation labelling and the reference data-sets are collected with identification of active or inactive (idle) state.

The initial reference data-set 410 may be generated by artificial data massage of artificially collected data. The generation of the initial reference data-set 410 is implemented by artificially collecting image/video data of hair/skin and performing artificial learning process, that is, converting collected data to reference data-set for recognition. When collecting the image/video data for the generation of the initial reference data-set 410, the microscope unit 100 may be applied to various positions of the hair or skin so that an appropriate data-set model can be selected and applied in the service field. The initial reference data-set 410 may be generated according to the resolution and magnification of the microscope unit 100.

The dynamic self-learning reference data-set 420 may be generated by automatically collecting or generating data with a software installed in the digital signage system with hair/skin care diagnosis function 10.

During the process of generating the dynamic self-learning reference data-set 420, the status information adapted to the resolution and magnification of the microscope unit 100 and brightness of the internal lighting of the service field with the digital signage system with hair/skin care diagnosis function 10 installed are generated by a software capable of automatically generating the hair/skin image and video data and self-learning using the data. By applying the results to the status information database of the service field, self-learning files are continuously updated without additional generation of the artificial data and learning.

The function of the image/video recognition on the hair/skin enables to recognize the input status by analyzing the status descriptor from the image and video data inputted from the microscope unit 100 and self-learns the status and recognizes the status of the hair/skin accurately.

The hair/skin image/video recognition software categorizes all the status descriptors of the image/video data into N representative status descriptor type. The hair/skin image/video recognition software analyzes each status descriptors of the hair/skin image/video data according to the N representative status descriptor and categorizes it as the most similar representative status descriptor type. The representative status descriptor can be selected by statistically analyzing the similarity of various types of status descriptors. For example, by applying a K-mean clustering algorithm, various types of status descriptors may be categorized into N representative type. The status of the hair/skin may be recognized by categorizing the inputted image/video data as one of the N representative type.

The hair/skin image/video recognition software of the digital signage system with hair/skin care diagnosis function 10 stores the dynamic self-learning status type, that is, the representative status descriptor and the corresponding hair/skin image/video status probability into the status information database. By integrating the results generated by the dynamic self-learning into the reference data-set status information database, the digital signage system with hair/skin care diagnosis function 10 can provide a service with a high degree of performance without additional artificial data generation and learning. In addition, the intelligence of the hair/skin status recognition of the digital signage system with hair/skin care diagnosis function 10 can be grown up continuously and a high degree of performance can be achieved by continuously updating the self-learning data-set.

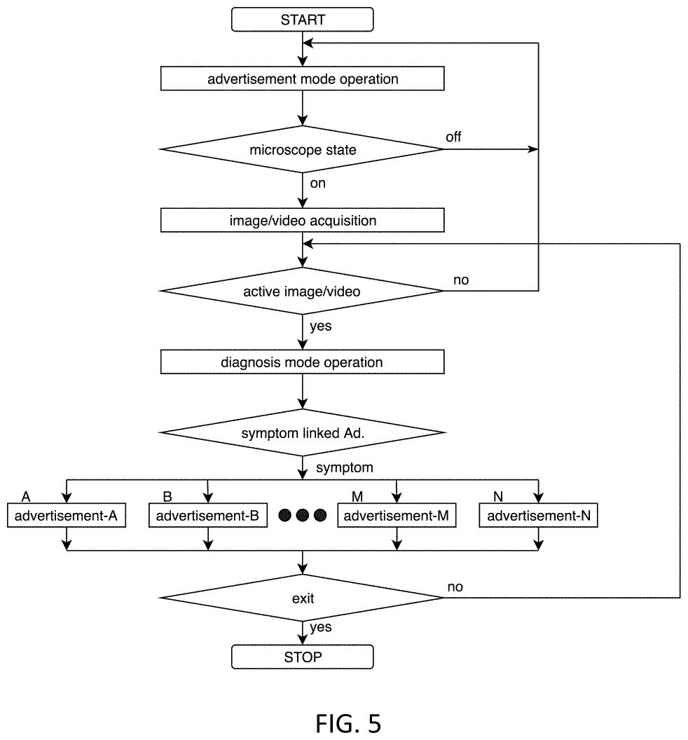

FIG. 5 is a schematic diagram illustrating a scenario of operation of the digital signage system with hair/skin care diagnosis function 10 according to an embodiment of the present invention.

Referring to FIG. 5, the digital signage system with hair/skin care diagnosis function 10 according to an embodiment of the present invention initially operates in the advertisement mode. The process unit 200 of the digital signage system with hair/skin care diagnosis function 10 monitors a connection state of the microscope unit. If the connection state of the microscope unit 100 is off, the digital signage system with hair/skin care diagnosis function 10 continues to operate in the advertisement mode. But, if the connection state of the microscope unit 100 is on, the image/video acquisition is performed. Then, the process unit 200 determines whether the image/video data received from the microscope unit 100 is active or inactive and, when the image/video data is active, the digital signage system with hair/skin care diagnosis function 10 begins to operate in the diagnosis mode. That is, the mode of operation of the digital signage system with hair/skin care diagnosis function 10 is automatically switched from the normal mode to the diagnosis mode when the image and video data is active.

Then, the image/video recognition part 220 of the process unit 200 analyzes the status of image and video data received from the image/video acquisition part 210, recognizes symptoms on hair and skin of the customer, quantifies the recognition results, and stores the quantification results in a symptom category information table which includes symptoms categorized according to various elements of the hair/skin, such as texture, blemishes, pores, brightness, hair loss, hair weakness, hair skin trouble and coloring. Detailed information on the process and the results of the image/video recognition is transmitted to the diagnosis analysis part 230 and summarized information is transmitted to the symptom-linked advertisement handling part 235.

In the above scenario, the mode of operation of the digital signage system with hair/skin care diagnosis function 10 is automatically switched from the normal mode to the diagnosis mode when the image and video data is valid. However, in another embodiment, the mode of operation of the digital signage system with hair/skin care diagnosis function 10 may be automatically switched from the normal mode to the diagnosis mode when the connection state of the microscope unit 100 is on. In another embodiment, the mode of operation of the digital signage system with hair/skin care diagnosis function 10 may be automatically switched from the normal mode to the diagnosis mode when the image and video data is valid, without the procedure of checking the state of the microscope unit 100.

In the foregoing descriptions, although the present invention has been described in connection with the specific matters, such as the specific components, the specific embodiments, and the drawings, they are provided only for assisting in the understanding of the present invention, and the present invention is not limited to the embodiments. It will be apparent that those skilled in the art can make various modifications and changes thereto from these descriptions.

Therefore, the spirit of the present invention should not be limited to the aforementioned embodiments, and the appended claims and what are modified equally or equivalently thereto will be considered to fall within the scopes of the present invention.

What is claimed is:

1. A digital signage system with hair/skin care diagnosis function comprising:
a microscope unit configured to scan hair/skin of a user and acquires hair/skin image and video data,
a process unit coupled to the microscope unit, and configured to process the hair/skin image and video data, and
a display unit coupled to the process unit, and configured to display a plurality of windows for displaying advertisements or the hair/skin image and video data and diagnosis results,
wherein the microscope unit controls magnification and resolution of the hair/skin image and video data, and transmits the hair/skin image and video data to the process unit, and
the process unit monitors a connection state of the microscope unit, receives the hair/skin image and video data from the microscope unit, stores the hair/skin image and video data, and switches a mode of operation of the digital signage system with hair/skin care diagnosis function from a normal mode to a diagnosis mode when the microscope unit operates and acquires the hair/skin image and video data, wherein in the normal mode, the process unit transmits advertisement contents to the display unit so that the plurality of windows display advertisements, and in the diagnosis mode, the process unit transmits both advertisement contents and the hair/skin image and video data to the display unit so that one of the plurality of windows displays the hair/skin image and video data and the diagnosis results, and another window of the plurality of windows displays advertisements.

2. The digital signage system of claim 1, wherein the process unit comprises:
a microscope interface part configured to manage a connection to the microscope unit, monitor on/off state of the microscope unit, and receive the hair/skin image and video data from the microscope unit,
a data-set management part configured to store and manage, for image/video recognition, a reference data-set consisting of a static reference image/video data-set and a dynamic reference image/video data-set, wherein the static reference image/video data-set is an initial and factory implemented data-set and the dynamic reference image/video data-set is a reference data-set obtained by processing the hair/skin image and video data collected from service locations where the digital signage system with hair/skin care diagnosis function is operated,
a communication and device handling part configured to receive the advertisement contents or provide the diagnosis results to the user through communication with a client node or an external server,
a power management part configured to manage the power that is supplied to the display unit and the microscope unit, and
an advertisement handling part configured to manage and operate the advertisement contents which are performed in the normal mode.

3. The digital signage system of claim 2, wherein the process unit further comprises:
a display management part configured to switch from the normal mode to the diagnosis mode and vice versa, according to on/off status of the microscope unit or valid/idle status of the image/video data inputted from the microscope unit, and manage and operate the plurality of windows of the display unit for each mode,
an image/video acquisition part configured to store the image and video data for the user's hair/skin care diagnosis in a file format into a memory allotted for the user, transmit to an image/video recognition part after converting the image and video data into a file format effective for the image/video recognition, monitor whether the image and video data is valid or idle, and transmit a valid/idle status to the display management part, to automatically switch the mode of operation of the digital signage system from the normal mode to the diagnosis mode,
the image/video recognition part configured to analyze the status of the image and video data received from the image/video acquisition part, recognize symptoms on hair and skin of the user, quantify recognition results, store the quantification results in a symptom category information table which includes symptoms categorized according to various elements of the hair/skin, such as texture, blemishes, pores, brightness, hair loss, hair weakness, hair skin trouble and coloring, and transmit detailed information on the quantification results of the image/video recognition to a diagnosis analysis part,
the diagnosis analysis part configured to store the detailed information on the quantification results in a database managing a history of hair and skin diagnosis of the user, analyze a status of the diagnosis and degree of progress in hair/skin care based on an accumulated data, process the analyzed information into a useful format to the customer, and provide the processed information, and
a symptom-linked advertisement handling part configured to manage and operate the advertisement contents which are performed in the diagnosis mode, display products or service related to the symptom of the user in an advertising window according to summarized information of the image/video recognition received from the image/video recognition part while the user is taking a hair/skin care or diagnosis.

4. The digital signage system of claim 1, wherein the diagnosis results are provided to the user via a mobile app.

5. A method for implementing the digital signage system with hair/skin care diagnosis function, the digital signage system with hair/skin care diagnosis function including a microscope unit configured to scan hair/skin of a user and acquires hair/skin image and video data, a process unit coupled to the microscope unit, and configured to process the hair/skin image and video data, and a display unit coupled to the process unit, and configured to display a plurality of windows for displaying advertisements or the hair/skin image and video data and diagnosis results, the method comprising:
a first operation in which the microscope unit controls magnification and resolution of the hair/skin image and video data, and transmits the hair/skin image and video data to the process unit; and
a second operation in which the process unit monitors a connection state of the microscope unit, receives the hair/skin image and video data from the microscope unit, stores the hair/skin image and video data, and switches a mode of operation of the digital signage system with hair/skin care diagnosis function from a normal mode to a diagnosis mode when the microscope unit operates and acquires the hair/skin image and video data, wherein in the normal mode, the process unit transmits advertisement contents to the display unit so that the plurality of windows display advertisements, and in the diagnosis mode, the process unit transmits both advertisement contents and the hair/skin image and video data to the display unit so that one of the plurality of windows displays the hair/skin image and video data and the diagnosis results, and another window of the plurality of windows displays advertisements.

6. The method of claim 5, wherein the process unit comprises:
a microscope interface part configured to manage a connection to the microscope unit, monitor on/off state of the microscope unit, and receive the hair/skin image and video data from the microscope unit,
a data-set management part configured to store and manage, for image/video recognition, a reference data-set consisting of a static reference image/video data-set and a dynamic reference image/video data-set, wherein the static reference image/video data-set is an initial and factory implemented data-set and the dynamic reference image/video data-set is a reference data-set obtained by processing the hair/skin image and video data collected from service locations where the digital signage system with hair/skin care diagnosis function is operated,
a communication and device handling part configured to receive the advertisement contents or provide the diagnosis results to the user through communication with a client node or an external server,
a power management part configured to manage the power that is supplied to the display unit and the microscope unit, and
an advertisement handling part configured to manage and operate the advertisement contents which are performed in the normal mode.

7. The method of claim 6, wherein the process unit further comprises:
a display management part configured to switch from the normal mode to the diagnosis mode and vice versa, according to on/off status of the microscope unit or valid/idle status of the image/video data inputted from the microscope unit, and manage and operate the plurality of windows of the display unit for each mode,
an image/video acquisition part configured to store the image and video data for the user's hair/skin care diagnosis in a file format into a memory allotted for the user, transmit to an image/video recognition part after converting the image and video data into a file format effective for the image/video recognition, monitor whether the image and video data is valid or idle, and transmit a valid/idle status to the display management part, to automatically switch the mode of operation of the digital signage system from the normal mode to the diagnosis mode,
the image/video recognition part configured to analyze the status of the image and video data received from the image/video acquisition part, recognize symptoms on hair and skin of the user, quantify recognition results, store the quantification results in a symptom category information table which includes symptoms categorized according to various elements of the hair/skin, such as texture, blemishes, pores, brightness, hair loss, hair weakness, hair skin trouble and coloring, and transmit detailed information on the quantification results of the image/video recognition to a diagnosis analysis part,
the diagnosis analysis part configured to store the detailed information on the quantification results in a database managing a history of hair and skin diagnosis of the user, analyze a status of the diagnosis and degree of progress in hair/skin care based on an accumulated data, process the analyzed information into a useful format to the customer, and provide the processed information, and
a symptom-linked advertisement handling part configured to manage and operate the advertisement contents which are performed in the diagnosis mode, display products or service related to the symptom of the user in an advertising window according to summarized information of the image/video recognition received from the image/video recognition part while the user is taking a hair/skin care or diagnosis.

8. The method of claim 5, wherein the diagnosis results are provided to the user via a mobile app.
